(19) United States
(12) Patent Application Publication
(10) Pub. No.: US 2016/0073976 A1
(43) Pub. Date: Mar. 17, 2016

(54) NUCLEAR MEDICINE DIAGNOSTIC APPARATUS AND NUCLEAR MEDICINE IMAGE GENERATING METHOD

(71) Applicants: Kabushiki Kaisha Toshiba, Minato-Ku (JP); Toshiba Medical Systems Corporation, Otawara-Shi (JP)

(72) Inventor: Kenta MORIYASU, Nasushiobara (JP)

(73) Assignees: Kabushiki Kaisha Toshiba, Minato-Ku (JP); Toshiba Medical Systems Corporation, Otawara-Shi (JP)

(21) Appl. No.: 14/944,432

(22) Filed: Nov. 18, 2015

Related U.S. Application Data
(63) Continuation of application No. PCT/JP2014/063716, filed on May 23, 2014.

(30) Foreign Application Priority Data
May 23, 2013 (JP) 2013-109258

(51) Int. Cl. A61B 6/03; A61B 6/00

(52) U.S. Cl. CPC A61B 6/037; A61B 6/5205; A61B 6/461

(57) ABSTRACT

According to one embodiment, a nuclear medicine diagnostic apparatus includes a processing circuitry. The processing circuitry acquires coincidence count data indicating an occurrence position of each of coincidentally counted pair-annihilation events, based on pieces of output data of a plurality of detectors that detect gamma rays emitted from radio isotopes administered to an object. Further, the processing circuitry generates, each time a condition necessary for a filter process is satisfied, a filter image by performing the filter process on the coincidence count data.

### CROSS-REFERENCE TO RELATED APPLICATIONS

This application is a Continuation application of No. PCT/JP2014/63716, filed on May 23, 2014, and the PCT application is based upon and claims the benefit of priority from Japanese Patent Application No. 2013-109258, filed on May 23, 2013, the entire contents of which are incorporated herein by reference.

### FIELD

Embodiments described herein relate generally to a nuclear medicine diagnostic apparatus and a nuclear medicine image generating method.

### BACKGROUND

Nuclear medicine diagnostic apparatuses such as a positron emission tomography (PET) apparatus use a property that a drug (a bloodstream marker, a tracer) containing radio isotopes (hereinafter, referred to as RIs) is selectively taken into a particular tissue or organ in a living body, and detect gamma rays emitted from the RIs distributed in the living body by means of gamma ray detectors provided outside of the living body. Detection results of the gamma rays are used to generate a nuclear medicine image by creating an image of dose distribution of the gamma rays and diagnose a function of the organ in the body.

In recent years, a TOF-PET apparatus is being developed. The TOF-PET apparatus obtains an occurrence position of an annihilation event on a line of response (LOR), on the basis of a difference in time of flight (TOF) between a pair of coincidentally counted pair-annihilation gamma rays. According to the TOF-PET apparatus of this type, an image of dose distribution of gamma rays can be created more accurately than conventional PET apparatuses.

However, because a speed of a gamma ray is a speed of light, an error in time of flight (TOF) resulting from a temporal resolution (for example, 500 psec) of a detector system cannot be ignored. Hence, in general, each of pieces of occurrence position information (hereinafter, referred to as position information) on annihilation events obtained from coincidence count information is subjected to a position filter such as a Gaussian filter. As a result, the occurrence position of each annihilation event is blurred by the position filter, and hence, even if a nuclear medicine image is reconstructed using only one piece of coincidence count information, it is difficult for a user to accurately understand the occurrence position of each annihilation event from this image.

A conceivable method for generating a nuclear medicine image that enables the user to accurately understand the occurrence position of each annihilation event includes accumulating a large number of pieces of coincidence count information and then reconstructing a nuclear medicine image.

For example, in a case where an adopted scan method is a mode of performing scanning while gradually changing a relative position relation between a bed and detectors (hereinafter, referred to as a continuous scan mode), a conceivable method includes: accumulating pieces of coincidence count information up to an end of the scanning; and reconstructing a nuclear medicine image on the basis of the accumulated pieces of coincidence count information after the end of the scanning. In a case where an adopted scan method is a mode of repeating a procedure of: moving one of the bed and the detectors to a next scan position upon an end of scanning at one scan position; and performing next scanning (hereinafter, referred to as a multi-head scan mode), a conceivable method includes reconstructing a nuclear medicine image on the basis of accumulated pieces of coincidence count information each time the scanning at one scan position is ended.

Unfortunately, these methods take time from an end of scanning to presentation of a nuclear medicine image corresponding to the scanning to the user, and, by the time the user can check the nuclear medicine image, the scanning at a position corresponding to this image has already been ended.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram illustrating an example of a nuclear medicine diagnostic apparatus according to a first embodiment of the present invention;

FIG. 2 is a schematic block diagram illustrating a function example implemented by the processor of the processing circuitry according to the first embodiment;

FIG. 3A is an explanatory view illustrating an example real-time image in a case where a temporal resolution of the TOF-PET apparatus is assumed to be ideally infinitesimally small;

FIG. 3B is an explanatory view illustrating an example real-time image in a case where the temporal resolution of the TOF-PET apparatus has a finite value;

FIG. 4 is an explanatory view illustrating an example method of using the real-time image memory circuitry, the filter image memory circuitry, and the display image memory circuitry in a case where a real-time image and a filter image are displayed on the display in a superimposed manner;

FIG. 5 is an explanatory view illustrating an example method of using the real-time image memory circuitry, the filter image memory circuitry, and the display image memory circuitry in a case where only a filter image is displayed on the display;

FIG. 6 is a flowchart illustrating a procedure when the processor of the processing circuitry illustrated in FIG. 1 generates, in real time, a nuclear medicine image that enables the user to understand an occurrence position of an annihilation event;

FIG. 7 is a schematic block diagram illustrating a function example implemented by a processor of a processing circuitry according to the second embodiment;

FIG. 8 is a flowchart illustrating a procedure when the processor of the processing circuitry illustrated in FIG. 7 generates, in real time, a nuclear medicine image that enables the user to understand an occurrence position of an annihilation event;

FIG. 9 is a schematic block diagram illustrating a function example implemented by a processor of a processing circuitry according to the third embodiment; and

FIG. 10 is a flowchart illustrating a procedure when the processor of the processing circuitry illustrated in FIG. 9 generates, in real time, a nuclear medicine image that enables the user to understand an occurrence position of an annihilation event through a high-speed image reconstructing process.

### DETAILED DESCRIPTION

Hereinbelow, a description will be given of a nuclear medicine diagnostic apparatus and a nuclear medicine image generating method according to embodiments of the present invention with reference to the drawings.

In general, according to one embodiment, a nuclear medicine diagnostic apparatus includes a processing circuitry. The processing circuitry acquires coincidence count data indicating an occurrence position of each of coincidentally counted pair-annihilation events, based on pieces of output data of a plurality of detectors that detect gamma rays emitted from radio isotopes administered to an object. Further, the processing circuitry generates, each time a condition necessary for a filter process is satisfied, a filter image by performing the filter process on the coincidence count data.

### First Embodiment

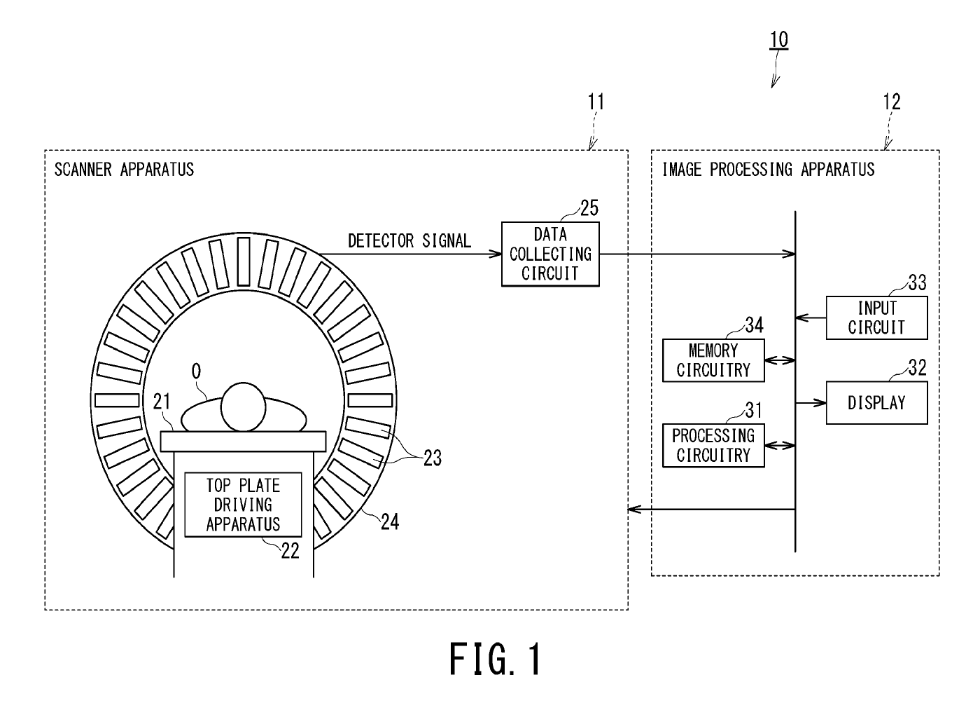

FIG. 1 is a block diagram illustrating an example of a nuclear medicine diagnostic apparatus according to a first embodiment of the present invention. In the following description, discussed is an example case where a TOF-PET apparatus is used as the nuclear medicine diagnostic apparatus according to the present invention.

The nuclear medicine diagnostic apparatus according to the present embodiment performs scanning in a multi-head scan mode. In the multi-head scan mode, a procedure of: moving a top plate to a next scan position upon an end of scanning at one scan position; and performing next scanning is repeated.

A nuclear medicine diagnostic apparatus 10 includes a scanner apparatus 11 and an image processing apparatus 12. The scanner apparatus 11 includes a top plate 21, a top plate driving apparatus 22, a plurality of detectors 23, a detector cover 24, and a data collecting circuit 25.

A patient (object) 0 can be placed on the top plate 21. The top plate driving apparatus 22 is controlled by the image processing apparatus 12 to move the top plate 21 up and down. The top plate driving apparatus 22 is controlled by the image processing apparatus 12 to transport the top plate 21 to an opening area in a central portion of the detector cover 24 along a long axis direction of the top plate 21.

Each detector 23 is a detector that detects gamma rays emitted from RIs that are contained in a drug such as fluorodeoxyglucose (FDG) and are administered to the patient O. A scintillator detector may be used as the detector 23, and a semiconductor detector may be used thereas.

In a case of using the scintillator detector, the detector 23 includes: a collimator for defining an entrance angle of a gamma ray; a scintillator that emits an instantaneous flash when a collimated gamma ray enters; a plurality of two-dimensionally arranged photomultiplier tubes for detecting light emitted from the scintillator; and an electronic circuit for the scintillator.

The scintillator is made of, for example, thallium-activated sodium iodide NaI(T1). Each time an event of gamma ray entrance occurs, the electronic circuit for the scintillator generates entrance position information (position information) and intensity information on gamma rays within a detection plane formed by the plurality of photomultiplier tubes, on the basis of outputs of the plurality of photomultiplier tubes, and outputs the generated information to the data collecting circuit 25.

In the case of using the semiconductor detector, the detector 23 includes: a collimator; a plurality of two-dimensionally arranged gamma ray detecting semiconductor elements (hereinafter, referred to as semiconductor elements) for detecting a collimated gamma ray; and an electronic circuit for the semiconductor.

Each semiconductor element is made of, for example, CdTe or CdZnTe (CZT). Each time an event of gamma ray entrance occurs, the electronic circuit for the semiconductor generates position information and intensity information on the basis of outputs of the semiconductor elements, and outputs the generated information to the data collecting circuit 25.

The plurality of detectors 23 are arranged in a hexagonal shape or a circular shape inside of the detector cover 24 so as to surround the patient O, for example. How to arrange the plurality of detectors 23 is not limited to the ring-like arrangement, and may be, for example, two-detector-group opposing arrangement. In the two-detector-group opposing arrangement, two groups of the plurality of detectors 23 respectively arranged on flat plates are arranged so as to be opposed to each other with the patient O being sandwiched therebetween, and are rotatably held around the patient O. The plurality of detectors 23 may be arranged in multi-layer rings so as to be capable of acquiring images between adjacent layers.

The data collecting circuit 25 includes at least a processor and a memory circuitry. A processing circuitry of the data collecting circuit 25 collects outputs of the plurality of detectors 23 in the form of list mode data or map image data of a photomultiplier tube, in accordance with a program stored in the memory circuitry. In the list mode, detection position information on a gamma ray, intensity (energy) information, information indicating a relative position between the detectors 23 and the patient O (a position and angle of each detector 23), and detection time of the gamma ray are collected each time a gamma ray annihilation event occurs.

The data collecting circuit 25 may collect the outputs of the plurality of detectors 23 in the form of coincidence list mode data. The coincidence list mode data is obtained by extracting, from list mode data, combinations that satisfy conditions that: an entrance time difference between gamma rays (a detection time difference between annihilation gamma rays) is within a predetermined time window width (for example, within 1 ns); and respective entrance energies of the two annihilation gamma rays are within a predetermined energy window width.

Hereinafter, an annihilation event corresponding to each of the extracted combinations is referred to as a coincidentally counted pair-annihilation event (coincidence count event). Data indicating an occurrence position of a coincidentally counted annihilation event obtained from coincidence list mode data is referred to as coincidence count data.

In the present embodiment, description is given of an example case where the occurrence position of the coincidentally counted pair-annihilation event obtained from the coincidence list mode data is subjected to a position filter such as a Gaussian filter. The position filter is applied considering an error in time of flight (TOF) resulting from a temporal resolution (for example, 500 psec) of a detector system. By blurring the position, the position information can be modified to information for which a measurement error in TOF is considered.

Specifically, a coordinate point of the occurrence position of the coincidence count event is obtained from a difference in measurement time between two detectors that detect the coincidence count event, and distribution that is blurred using a Gaussian function corresponding to a temporal resolution of the detectors along a line of response (LOR) is defined as the position information. Hence, in the present embodiment, the occurrence position of the annihilation event indicated by the coincidence count data is given as a line segment having a predetermined length along the LOR.

As illustrated in FIG. 1, the image processing apparatus 12 includes a processing circuitry 31, a display 32, an input circuit 33, and a memory circuitry 34.

The processing circuitry 31 includes at least a processor, is configured using, for example, the processor, a RAM, and a memory medium typified by a ROM, and controls a processing operation of the image processing apparatus 12 in accordance with programs stored in the memory medium.

The processor of the processing circuitry 31 loads, onto the RAM, a nuclear medicine image generating program and data necessary to execute this program, which are stored in the memory medium typified by the ROM, and executes a process for generating, in real time, a nuclear medicine image that enables a user to understand an occurrence position of an annihilation event, in accordance with this program.

The RAM of the processing circuitry 31 provides a work area for temporarily storing programs and data executed by the processor. The memory medium typified by the ROM of the processing circuitry 31 stores an activation program of the image processing apparatus 12, the nuclear medicine image generating program, and various pieces of data necessary to execute these programs.

The memory medium typified by the ROM may include a recording medium readable by the processor, such as a magnetic or optical recording medium or a semiconductor memory, and the entirety or a part of the programs and the data in the memory medium may be downloaded via an electronic network.

The display 32 is configured using, for example, general display/output apparatuses such as a liquid crystal display and an organic light emitting diode (OLED) display, and displays various pieces of information on a real-time image, a filter image, and other images in accordance with control of the processing circuitry 31.

The input circuit 33 is configured using, for example, general input apparatuses such as a keyboard, a touch panel, and a numeric keypad, and outputs an operation input signal corresponding to an operation by the user, to the processing circuitry 31.

The memory circuitry 34 may include a recording medium readable by the processor, such as a magnetic or optical recording medium or a semiconductor memory, and the entirety or a part of the programs and the data in the memory medium may be downloaded via an electronic network. The memory circuitry 34 is controlled by the processing circuitry 31 to store coincidence list mode data and a real-time image, a filter image, and other images generated on the basis of the coincidence list mode data.

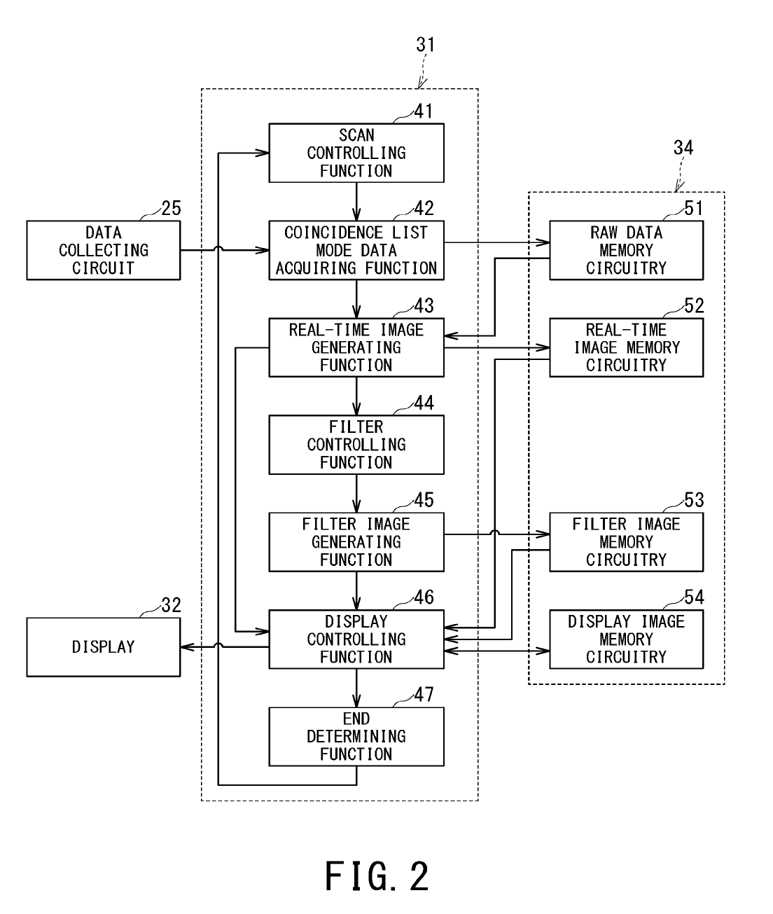

FIG. 2 is a schematic block diagram illustrating a function example implemented by the processor of the processing circuitry 31 according to the first embodiment.

As illustrated in FIG. 2, the nuclear medicine image generating program causes the processor of the processing circuitry 31 to function as at least a scan controlling function 41, a coincidence list mode data acquiring function 42, a real-time image generating function 43, a filter controlling function 44, a filter image generating function 45, a display controlling function 46, and an end determining function 47. These functions are each stored in the memory circuitry in the form of a program.

As illustrated in FIG. 2, the memory circuitry 34 includes a raw data memory circuitry 51, a real-time image memory circuitry 52, a filter image memory circuitry 53, and a display image memory circuitry 54. The raw data memory circuitry 51 and the memory circuitries 52 to 54 may be memory media physically independent of one another, and two (for example, the real-time image memory circuitry 52 and the filter image memory circuitry 53) or more thereof may be virtually allotted to a memory space of one memory medium.

The scan controlling function 41 receives an instruction to execute a scan plan from the user via the input circuit 33, and controls the scanner apparatus 11 on the basis of the scan plan, to thereby execute scanning. As a result, information on gamma rays emitted from the patient O is given to the coincidence list mode data acquiring function 42 from the scanner apparatus 11 via the data collecting circuit 25. The scan plan in the present embodiment is a scan plan according to the multi-head scan mode in which a procedure of: moving the top plate 21 to a next scan position upon an end of scanning at one scan position; and performing next scanning is repeated.

If raw data received from the data collecting circuit 25 is not coincidence list mode data, the coincidence list mode data acquiring function 42 creates coincidence list mode data on the basis of the raw data received from the data collecting circuit 25, and stores the created data into the raw data memory circuitry 51. On the other hand, if the raw data is coincidence list mode data, the coincidence list mode data acquiring function 42 stores the raw data into the raw data memory circuitry 51 as it is. Even if the raw data received from the data collecting circuit 25 is not coincidence list mode data, the raw data may be stored into the raw data memory circuitry 51 as it is, and the raw data may be converted into coincidence list mode data before real-time image generation to be described later (after coincidence list mode data is read out of the raw data memory circuitry 51).

In the following description, the coincidence list mode data that is stored into the raw data memory circuitry 51 by the coincidence list mode data acquiring function 42 is also referred to as raw data as appropriate, similarly to pieces of output data of the plurality of detectors 23 that are received from the data collecting circuit 25.

The data collecting circuit 25 collects outputs of the plurality of detectors 23 for each annihilation event of gamma rays, and gives the outputs to the processing circuitry 31. Hence, the coincidence list mode data acquiring function 42 can update the coincidence list mode data for each coincidentally counted pair-annihilation event. The coincidence list mode data acquiring function 42 can acquire, for each coincidence count event, data indicating an occurrence position of the coincidence count event (coincidence count data) on the basis of the coincidence list mode data.

Each time the coincidence list mode data acquiring function 42 acquires coincidence count data (data indicating an occurrence position of a coincidentally counted pair-annihilation event (coincidence count event)), that is, each time a coincidence count event is detected, the real-time image generating function 43 generates an image (hereinafter, referred to as a real-time image) obtained by superimposing an image indicating an occurrence position of a coincidence count event, and stores the real-time image into the real-time image memory circuitry 52.

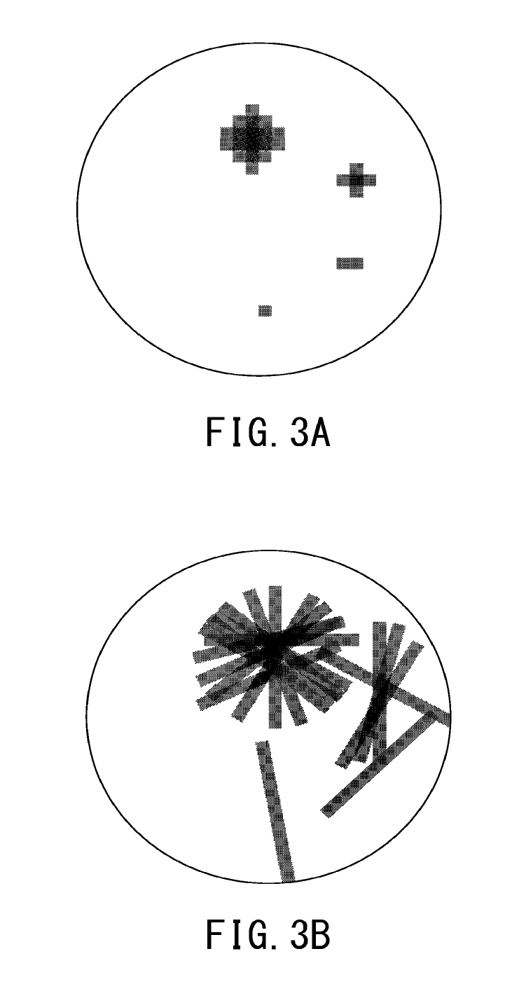

FIG. 3A is an explanatory view illustrating an example real-time image in a case where a temporal resolution of the TOF-PET apparatus is assumed to be ideally infinitesimally small, and FIG. 3B is an explanatory view illustrating an example real-time image in a case where the temporal resolution of the TOF-PET apparatus has a finite value.

In the case where the temporal resolution of the TOF-PET apparatus is assumed to be ideally infinitesimally small, as illustrated in FIG. 3A, each of images each indicating an occurrence position of a coincidence count event is infinitesimally close to a point, and the real-time image is an image in which these points are superimposed on each other.

In reality, the temporal resolution of the TOF-PET apparatus has a finite value (for example, 500 ps). Hence, as illustrated in FIG. 3B, the real-time image generated by the real-time image generating function 43 according to the present embodiment is an image in which each of images each indicating an occurrence position of a coincidence count event is expressed as a line segment having a predetermined length along a line of response (LOR), due to an influence of a position filter.

Hence, the real-time image according to the present embodiment is an image having a low spatial resolution of an occurrence position of a coincidence count event, and is an image that makes it difficult for the user to precisely judge the occurrence position of the coincidence count event, but is an image that enables the user to roughly understand the occurrence position of the coincidence count event. Accordingly, according to this real-time image, the user can understand the occurrence position of the coincidence count event more in real time, compared with a case of reconstructing a nuclear medicine image on the basis of accumulated pieces of coincidence count information each time scanning at one scan position is ended, in the multi-head scan mode.

The filter controlling function 44 determines whether or not a condition necessary for a filter process is satisfied. If determining that the condition necessary for the filter process is satisfied, the filter controlling function 44 instructs the filter image generating function 45 to perform the filter process on coincidence count data (or data on a real-time image that is an image generated by accumulating pieces of coincidence count data) and thus generate a filter image.

Examples of the filter process include a filter process used for filtered back projection. Various kinds of filter processes have been known up to now as the filter process used for filtered back projection, examples thereof include a filter process using a Shepp & Logan filter and a 2D or 3D filter process using a Ramp filter, and arbitrary one of these filter processes can be adopted.

Examples of the condition necessary for the filter process include a condition that a predetermined time has elapsed and a condition that a predetermined amount of counting has been accumulated. In a case where the condition that the predetermined amount of counting has been accumulated is used as the condition necessary for the filter process, the filter controlling function 44 may acquire, for example, information on the number of coincidence count events (the number of pieces of coincidence count data respectively corresponding to annihilation events) stored in the raw data memory circuitry 51, and may instruct the filter image generating function 45 to generate a filter image each time a predetermined number (for example, 20 counts) of events (pieces of data) are accumulated.

In the following description, discussed as appropriate is an example case where the filter controlling function 44 uses a condition that a predetermined time T1 has elapsed, as the condition necessary for the filter process and where the predetermined time T1 is a time T required for the filter process. It is sufficient that T1 be equal to or more than T, and T1 does not necessarily need to be equal to T.

Each time the predetermined time T1 (≧T) elapses (each time the time T required for the filter process elapses, in the case where T1=T, for example), the filter image generating function 45 receives an instruction to generate a filter image from the filter controlling function 44, performs the filter process on coincidence count data (or data on a real-time image that is an image generated by accumulating pieces of coincidence count data), thus generates a filter image, and stores the filter image into the filter image memory circuitry 53.

The display controlling function 46 expands at least one of a real-time image and a filter image in the display image memory circuitry 54, and displays the image(s) on the display 32. That is, on the display 32, the display controlling function 46 may display only the real-time image, may display only the filter image, may display the two images in a superimposed manner, and may simultaneously display the two images in different windows next to each other.

The end determining function 47 controls the scanner apparatus 11 via the scan controlling function 41 to execute scanning at a next scan position upon an end of scanning at one scan position. The end determining function 47 stops an operation of the scanner apparatus 11 via the scan controlling function 41 upon an end of scanning at every scan position.

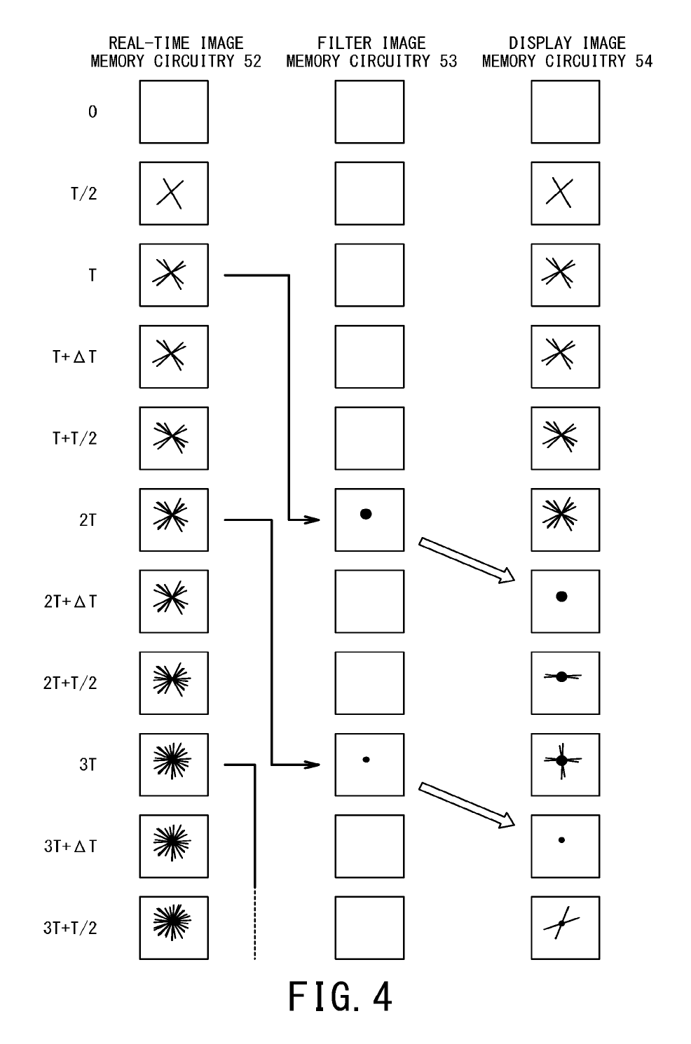

FIG. 4 is an explanatory view illustrating an example method of using the real-time image memory circuitry 52, the filter image memory circuitry 53, and the display image memory circuitry 54 in a case where a real-time image and a filter image are displayed on the display 32 in a superimposed manner.

As illustrated in a left column in FIG. 4, each time a coincidence count event is detected, the real-time image generating function 43 superimposes a line segment image indicating an occurrence position of the coincidence count event into the real-time image memory circuitry 52 one by one, to thereby update a real-time image.

In response to an elapse of a period during which a time t=0 to t=T1≧T (where T is the time required for the filter process), which is the first period, the filter image generating function 45 receives an instruction to generate a filter image from the filter controlling function 44. FIG. 4 illustrates an example case where T1=T. Then, the filter image generating function 45 performs the filter process on pieces of coincidence count data that are accumulated in the raw data memory circuitry 51 at the time t=T1 (or data on real-time images that are stored in the real-time image memory circuitry 52 at the time t=T1), and thus generates a filter image. A plurality of the filter image generating functions 45 may be provided. In this case, filter image generating processes can be simultaneously performed in parallel, and hence T1 may be set to be less than T. For example, in a case where two filter image generating functions 45 are provided, T1 may be set to be equal to or more than T/2.

Because the filter process requires the time T, the filter image generating function 45 stores the filter image corresponding to the coincidence count data in the period during which the time t=0 to t=T1 (≧T), into the filter image memory circuitry 53 at a time T1+T or after (for example, 2T1; in the example case where T1=T in FIG. 4, a time 2T) (see a middle column in FIG. 4). At the time 2T1 at which the time T1 has elapsed from the previous instruction to generate a filter image at the time T1, the filter image generating function 45 receives again an instruction to generate a filter image from the filter controlling function 44. In this way, each time the time T1 elapses, the filter image generating function 45 repeats a procedure of performing the filter process on coincidence count data and thus generating a filter image.

Each time the real-time image memory circuitry 52 is updated, the display controlling function 46 sequentially expands contents thereof in the display image memory circuitry 54. Moreover, if the filter image in the filter image memory circuitry 53 is updated, the display controlling function 46 expands contents thereof in the display image memory circuitry 54. At this time, the real-time image that has been expanded up to then in the display image memory circuitry 54 may be deleted. Alternatively, this real-time image may be left as it is, and the filter image may be superimposed thereon.

In a right column in FIG. 4, illustrated is an example case where the real-time image is deleted each time the filter image is expanded in the display image memory circuitry 54 and where a portion newly added after filter image update, of the real-time image is sequentially superimposed in the display image memory circuitry 54 each time the real-time image memory circuitry 52 is updated, in a period up to next filter image update.

A spatial resolution of the filter image is higher than that of the real-time image, and hence the filter image can more accurately indicate an occurrence position of a coincidence count event. Hence, the user can check an image with higher visual recognition properties. That is, if the filter image is displayed, the user can view an image with higher visual recognition properties, compared with a case where only the real-time image is displayed.

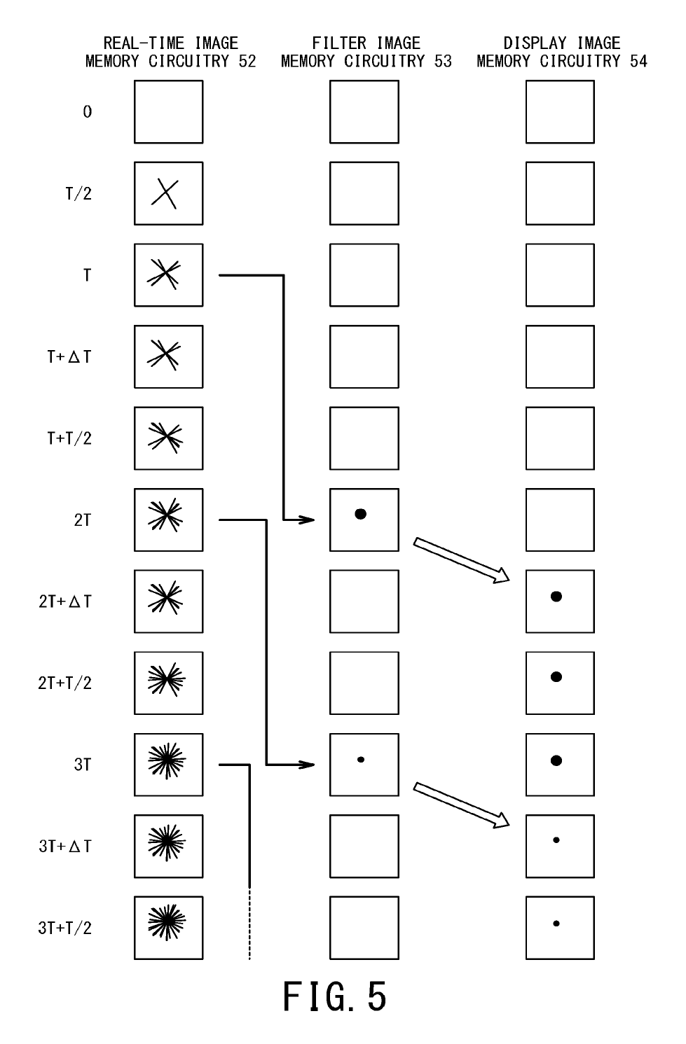

FIG. 5 is an explanatory view illustrating an example method of using the real-time image memory circuitry 52, the filter image memory circuitry 53, and the display image memory circuitry 54 in a case where only a filter image is displayed on the display 32.

As illustrated in FIG. 5, only a filter image may be displayed on the display 32. In this case, as illustrated in FIG. 5, the display controlling function 46 may expand only a filter image in the display image memory circuitry 54. A generation period of a filter image is the time T1≧T, and the time T1 is shorter than the time required for scanning at one scan position. Hence, even in the case where only a filter image is displayed, the user can check a filter image with a high spatial resolution more in real time, and can accurately understand an occurrence position of a coincidence count event in real time, compared with a case of reconstructing a nuclear medicine image on the basis of accumulated pieces of coincidence count information each time scanning at one scan position is ended, in the multi-head scan mode. As a matter of course, as described above, in a case where a plurality of the filter image generating functions 45 each having the filter process time T are provided, the time required for the filter process can be shortened by a parallel process, and hence T1 can be further shortened.

Next, an example operation of the nuclear medicine diagnostic apparatus and a nuclear medicine image generating method according to the present embodiment is described.

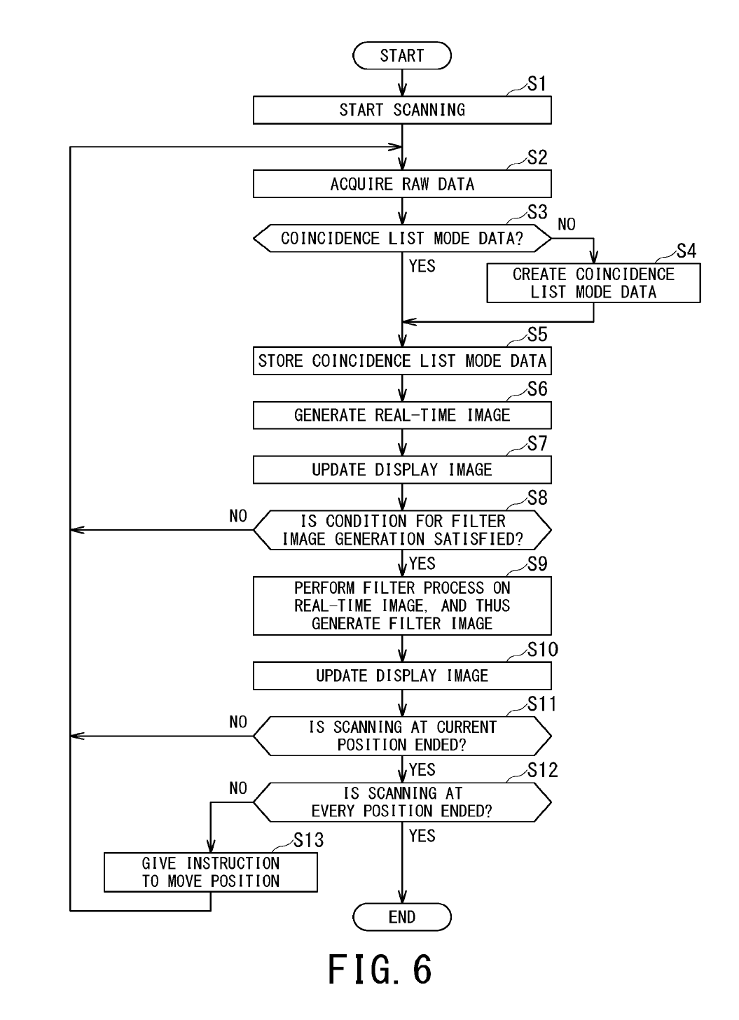

FIG. 6 is a flowchart illustrating a procedure when the processor of the processing circuitry 31 illustrated in FIG. 1 generates, in real time, a nuclear medicine image that enables the user to understand an occurrence position of an annihilation event. In FIG. 6, reference signs of S with a number respectively denote steps in the flowchart. In the following description, an example case where T1=T is discussed.

This procedure is started at the time at which the patient O to whom a drug such as FDG has been administered is placed on the top plate 21. In this procedure, description is given of an example case (see FIG. 4) where a real-time image and a filter image are displayed on the display 32 in a superimposed manner.

First, in Step S1, the scan controlling function 41 receives an instruction to execute a scan plan according to the multi-head scan mode from the user via the input circuit 33, and controls the scanner apparatus 11 on the basis of the scan plan, to thereby start scanning.

In Step S2, the coincidence list mode data acquiring function 42 receives pieces of output data (raw data) of the plurality of detectors 23 from the data collecting circuit 25.

In Step S3, the coincidence list mode data acquiring function 42 determines whether or not the raw data received from the data collecting circuit 25 is coincidence list mode data. If the received raw data is not coincidence list mode data, this procedure goes to Step S4. On the other hand, if the received raw data is coincidence list mode data, this procedure goes to Step S5.

In Step S4, the coincidence list mode data acquiring function 42 creates coincidence list mode data on the basis of the raw data received from the data collecting circuit 25.

In Step S5, the coincidence list mode data acquiring function 42 stores the coincidence list mode data (raw data) into the raw data memory circuitry 51. Even if it is determined in Step S3 that the raw data is not coincidence list mode data, the raw data may be stored into the raw data memory circuitry 51 as it is. In this case, the coincidence list mode data may be created after raw data storage and before real-time image generation in Step S6.

The data collecting circuit 25 collects outputs of the plurality of detectors 23 for each annihilation event of gamma rays, and gives the outputs to the processing circuitry 31. Hence, the coincidence list mode data acquiring function 42 can update the coincidence list mode data for each coincidentally counted pair-annihilation event. For this reason, Steps S2 to S5 may be executed in parallel with the following Step S6 and subsequent steps.

In Step S6, each time the coincidence list mode data acquiring function 42 acquires coincidence count data, that is, each time a coincidence count event is detected, the real-time image generating function 43 superimposes a line segment image indicating an occurrence position of the coincidence count event into the real-time image memory circuitry 52 one by one, to thereby generate a real-time image, and stores the real-time image into the real-time image memory circuitry 52.

In Step S7, the display controlling function 46 updates contents of the display image memory circuitry 54 in response to the update of the real-time image memory circuitry 52, to thereby update the image displayed on the display 32.

In Step S8, the filter controlling function 44 determines whether or not the condition necessary for the filter process used for filtered back projection is satisfied. For example, the filter controlling function 44 determines whether or not a cycle of the time T required for the filter process has come. If the condition necessary for the filter process is satisfied, the filter controlling function 44 gives the filter image generating function 45 an instruction to generate a filter image, and this procedure goes to Step S9. On the other hand, if the condition necessary for the filter process is not satisfied, this procedure returns to Step S2.

In Step S9, the filter image generating function 45 performs the filter process on coincidence count data (or data on a real-time image that is an image generated by accumulating pieces of coincidence count data), thus generates a filter image, and stores the filter image into the filter image memory circuitry 53.

In Step S10, the display controlling function 46 updates contents of the display image memory circuitry 54 in response to the update of the filter image memory circuitry 53, to thereby update the image displayed on the display 32.

In Step S11, the end determining function 47 determines whether or not scanning at a current scan position is ended. If the scanning at the current scan position is not ended, this procedure returns to Step S2.

On the other hand, if the scanning at the current scan position is ended, in Step S12, the end determining function 47 further determines whether or not scanning at every scan position is ended. If the scanning at every scan position is not ended, in Step S13, the end determining function 47 controls the scanner apparatus 11 via the scan controlling function 41 to execute scanning at a next scan position, and this procedure returns to Step S2. On the other hand, if the scanning at every scan position is ended, the end determining function 47 stops an operation of the scanner apparatus 11 via the scan controlling function 41, and this procedure is ended.

Through the above-mentioned procedure, a nuclear medicine image that enables the user to understand an occurrence position of an annihilation event can be generated in real time in the multi-head scan mode.

In the nuclear medicine diagnostic apparatus 10 according to the present embodiment, at least one of a real-time image and a filter image can be displayed on the display 32 in the multi-head scan mode. For example, in a case where the real-time image is displayed, the real-time image can be displayed on the display 32 while being updated, in a cycle much shorter than the time required up to an end of scanning at one scan position in the multi-head scan mode. Hence, the user can understand an occurrence position of a coincidence count event more in real time, compared with a case of reconstructing a nuclear medicine image on the basis of accumulated pieces of coincidence count information each time scanning at one scan position is ended, in the multi-head scan mode.

In a case where the filter image is displayed, the filter image having a higher spatial resolution than that of the real-time image can be displayed on the display 32 while being updated, in a cycle T1 much shorter than the time required up to an end of scanning at one scan position in the multi-head scan mode. Hence, the user can understand an occurrence position of a coincidence count event more in real time, compared with a case of reconstructing a nuclear medicine image on the basis of accumulated pieces of coincidence count information each time scanning at one scan position is ended, in the multi-head scan mode. Moreover, if the filter image is displayed, the user can more accurately understand an occurrence position of a coincidence count event on the basis of the filter image superior in spatial resolution, compared with a case where only the real-time image is displayed.

In a case where the condition that the time T required for the filter process has elapsed is used as the condition necessary for the filter process, a risk of stagnation in process can be prevented, and the filter process can be reliably executed.

### Second Embodiment

Next, a second embodiment of the nuclear medicine diagnostic apparatus and the nuclear medicine image generating method according to the present invention is described.

A nuclear medicine diagnostic apparatus according to the second embodiment is different from the nuclear medicine diagnostic apparatus according to the first embodiment in that scanning is performed according to a continuous scan mode in which an entirety of a photographing target site of an object is scanned while a relative position relation between a bed and the detectors 23 is gradually changed.

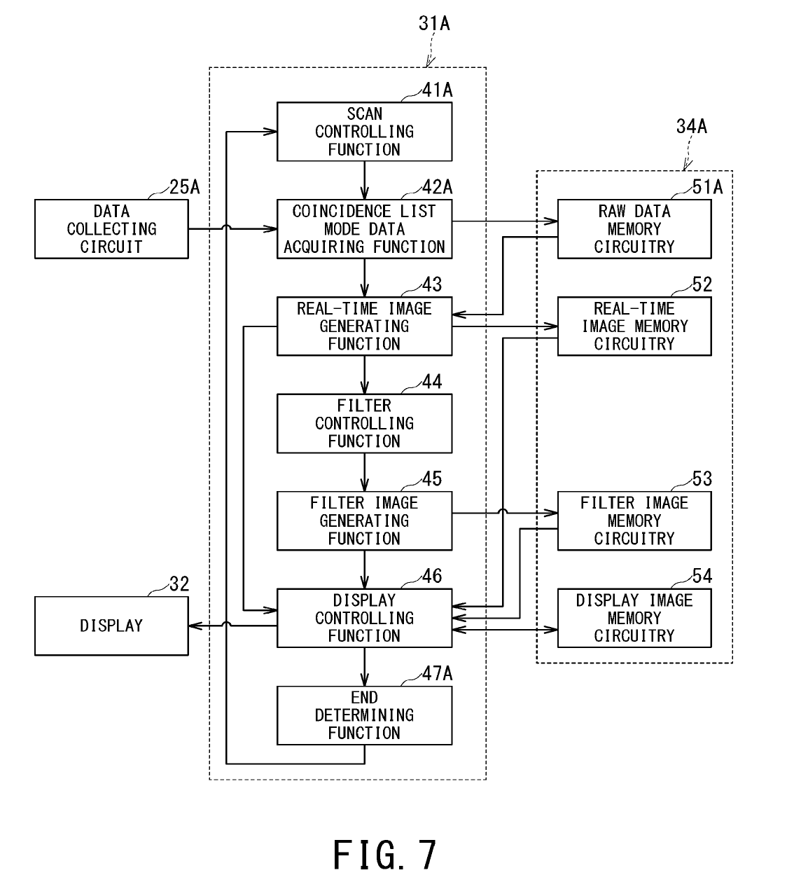

FIG. 7 is a schematic block diagram illustrating a function example implemented by a processor of a processing circuitry 31A according to the second embodiment.

In the nuclear medicine diagnostic apparatus 10 according to the second embodiment, configurations of a data collecting circuit 25A, the processing circuitry 31A, and a memory circuitry 34A are different from those of the data collecting circuit 25, the processing circuitry 31, and the memory circuitry 34 of the nuclear medicine diagnostic apparatus 10 according to the first embodiment. The other configurations and actions are not substantially different from those of the nuclear medicine diagnostic apparatus 10 illustrated in FIG. 1. Hence, the same configurations are denoted by the same reference signs, and description thereof is omitted.

As illustrated in FIG. 7, the nuclear medicine image generating program causes the processor of the processing circuitry 31A to function as at least a scan controlling function 41A, a coincidence list mode data acquiring function 42A, the real-time image generating function 43, the filter controlling function 44, the filter image generating function 45, the display controlling function 46, and an end determining function 47A. These functions are each stored in the memory circuitry in the form of a program.

The scan controlling function 41A receives an instruction to execute a scan plan according to the continuous scan mode from the user via the input circuit 33, and controls the scanner apparatus 11 on the basis of the scan plan, to thereby start scanning.

The coincidence list mode data acquiring function 42A receives raw data and bed position information that is information indicating a position of the top plate 21, from the data collecting circuit 25A.

In the continuous scan mode, a collection site of the object targeted by the plurality of detectors 23 gradually changes with time. Hence, the bed position information is necessary to obtain information indicating that outputs of the plurality of detectors 23 at each collection timing derive from gamma rays emitted from which position of the object.

If the raw data received from the data collecting circuit 25A is not coincidence list mode data, the coincidence list mode data acquiring function 42A creates coincidence list mode data on the basis of the raw data received from the data collecting circuit 25A, and stores the created data in association with the bed position information into a raw data memory circuitry 51A. On the other hand, if the raw data is coincidence list mode data, the coincidence list mode data acquiring function 42A stores the raw data and the bed position information received from the data collecting circuit 25A, in association with each other into the raw data memory circuitry 51A. Even if the raw data received from the data collecting circuit 25A is not coincidence list mode data, the raw data and the bed position information may be stored in association with each other into the raw data memory circuitry 51A, and the raw data may be converted into coincidence list mode data before real-time image generation.

The end determining function 47A stops an operation of the scanner apparatus 11 via the scan controlling function 41A upon an end of the scanning in the continuous scan mode.

Next, an example operation of the nuclear medicine diagnostic apparatus and a nuclear medicine image generating method according to the second embodiment is described.

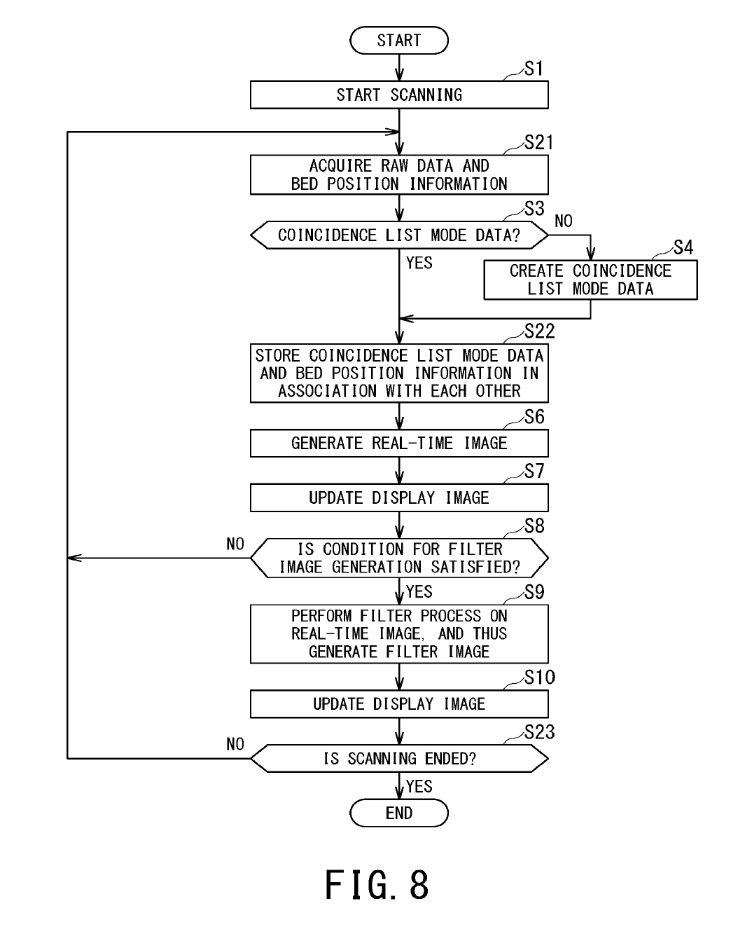

FIG. 8 is a flowchart illustrating a procedure when the processor of the processing circuitry 31A illustrated in FIG. 7 generates, in real time, a nuclear medicine image that enables the user to understand an occurrence position of an annihilation event. In FIG. 8, reference signs of S with a number respectively denote steps in the flowchart.

This procedure is started at the time at which the patient O to whom a drug such as FDG has been administered is placed on the top plate 21. In this procedure, description is given of an example case (see FIG. 4) where a real-time image and a filter image are displayed on the display 32 in a superimposed manner. Steps equivalent to those in FIG. 6 are denoted by the same reference signs, and overlapping description is omitted.

In Step S1, the scan controlling function 41A starts scanning according to the continuous scan mode. After that, in Step S21, the coincidence list mode data acquiring function 42A acquires raw data and bed position information from the data collecting circuit 25A.

In Step S22, the coincidence list mode data acquiring function 42A stores the coincidence list mode data (raw data) and the bed position information in association with each other into the raw data memory circuitry 51A. Even if it is determined in Step S3 that the raw data is not coincidence list mode data, the raw data and the bed position information may be stored as they are, in association with each other into the raw data memory circuitry 51A. In this case, the coincidence list mode data may be created after raw data storage and before real-time image generation in Step S6.

In Step S23, the end determining function 47A determines whether or not the scanning in the continuous scan mode is ended. If the scanning in the continuous scan mode is not ended, this procedure returns to Step S21. On the other hand, if the scanning in the continuous scan mode is ended, this procedure is ended.

Through the above-mentioned procedure, a nuclear medicine image that enables the user to understand an occurrence position of an annihilation event can be generated in real time in the continuous scan mode.

The nuclear medicine diagnostic apparatus 10 according to the present embodiment produces effects similar to those produced by the nuclear medicine diagnostic apparatus according to the first embodiment, even in the continuous scan mode.

### Third Embodiment

Next, a third embodiment of the nuclear medicine diagnostic apparatus and the nuclear medicine image generating method according to the present invention is described.

The nuclear medicine diagnostic apparatus according to the first embodiment and the nuclear medicine diagnostic apparatus according to the second embodiment each generate a real-time image by plotting detected coincidence count events in real time onto a real coordinate space, and generate a filter image by performing, for example, a 3D filter process on the real-time image. In comparison, a nuclear medicine diagnostic apparatus according to the third embodiment is a nuclear medicine diagnostic apparatus capable of executing an image reconstructing process at high speed, and successively generates and displays a reconstruction image as soon as a number of coincidence count events are accumulated, the number being necessary for the reconstructing process.

Here, the expression “being capable of executing a reconstructing process at high speed” refers to being capable of executing the reconstructing process in a time shorter than the time required for scanning at one scan position in the multi-head scan mode and in a time shorter than the time required for a series of scanning operations in the continuous scan mode.

In the following description, discussed is an example case where scanning according to the multi-head scan mode is performed, but the nuclear medicine diagnostic apparatus according to the third embodiment can generate a reconstruction image even in a case of scanning according to the continuous scan mode.

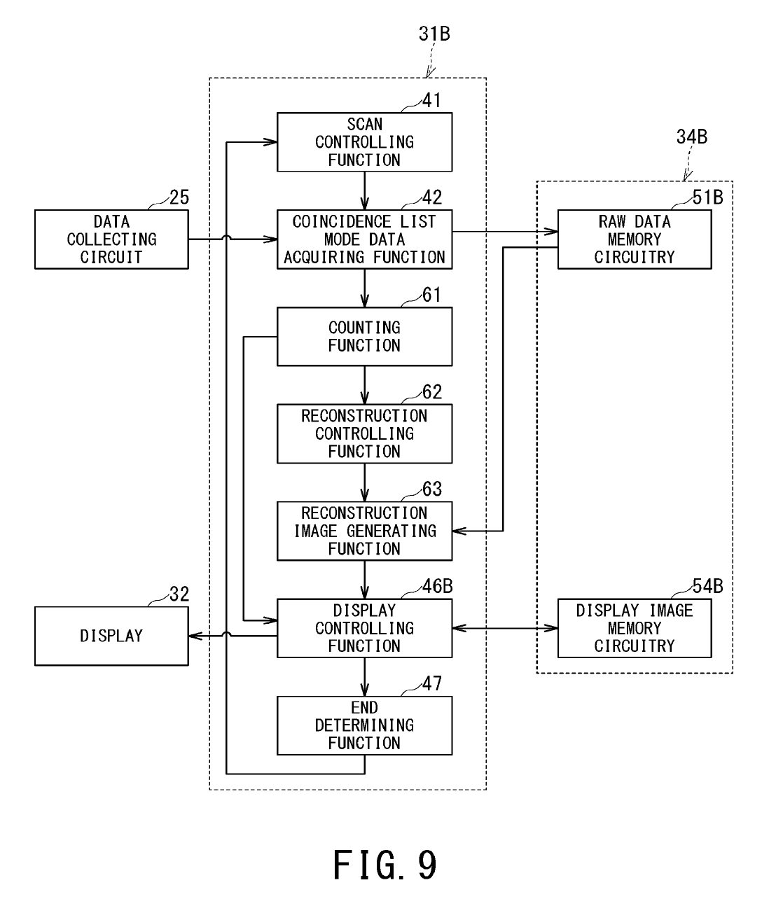

FIG. 9 is a schematic block diagram illustrating a function example implemented by a processor of a processing circuitry 31B according to the third embodiment.

In the nuclear medicine diagnostic apparatus 10 according to the third embodiment, configurations of the processing circuitry 31B and a memory circuitry 34B are different from those of the processing circuitry 31 and the memory circuitry 34 of the nuclear medicine diagnostic apparatus 10 according to the first embodiment. The other configurations and actions are not substantially different from those of the nuclear medicine diagnostic apparatus 10 illustrated in FIG. 1. Hence, the same configurations are denoted by the same reference signs, and description thereof is omitted.

As illustrated in FIG. 9, the nuclear medicine image generating program causes the processor of the processing circuitry 31B to function as at least the scan controlling function 41, the coincidence list mode data acquiring function 42, a counting function 61, a reconstruction controlling function 62, a reconstruction image generating function 63, a display controlling function 46B, and the end determining function 47. These functions are each stored in the memory circuitry in the form of a program.

The counting function 61 counts a count number of coincidence count events received from the data collecting circuit 25. In a case where the counting function 61 is not used by the reconstruction controlling function 62, the counting function 61 may not be provided.

The reconstruction controlling function 62 determines whether or not a condition necessary for an image reconstructing process is satisfied. If determining that the condition necessary for the image reconstructing process is satisfied, the reconstruction controlling function 62 instructs the reconstruction image generating function 63 to perform the image reconstructing process on the basis of coincidence count data and thus generate a reconstruction image.

Here, various kinds of methods have been known up to now as an image reconstructing method, examples thereof include filtered back projection and successive approximation, and arbitrary one of these methods can be adopted.

A condition indicating that an amount of data necessary for the image reconstructing process has been collected is preferably used as the condition necessary for the image reconstructing process, and examples thereof include a condition that a predetermined time has elapsed and a condition that a predetermined count number has been counted by the counting function 61.

A condition that the time that is required for the image reconstruction by the reconstruction image generating function 63 has elapsed may also be used as the condition necessary for the image reconstructing process. In this case, each time a time obtained by adding a predetermined time (including zero) to the time required for the image reconstruction elapses, the reconstruction image generating function 63 generates a reconstruction image. In this case, for example, coincidence count data collected within a period having a length equal to or less than the time required for the image reconstruction can be used as data for each reconstruction image.

Each time the condition necessary for the image reconstructing process is satisfied, the reconstruction image generating function 63 receives an instruction to generate a reconstruction image from the reconstruction controlling function 62, performs the image reconstructing process on the basis of coincidence count data (raw data stored in a raw data memory circuitry 51B), and thus generates a reconstruction image.

For example, in the multi-head scan mode, each time the condition necessary for the image reconstructing process is satisfied, the reconstruction image generating function 63 generates a reconstruction image in parallel with a data collecting process by the data collecting circuit 25 at the same scan position.

Each time a reconstruction image is generated by the reconstruction image generating function 63, the display controlling function 46B expands the reconstruction image in a display image memory circuitry 54B, and displays the reconstruction image on the display 32.

In a case of executing scanning according to the continuous scan mode, the data collecting circuit 25, the scan controlling function 41, the coincidence list mode data acquiring function 42, and the end determining function 47 may be respectively replaced with the data collecting circuit 25A, the scan controlling function 41A, the coincidence list mode data acquiring function 42A, and the end determining function 47A according to the second embodiment.

Next, an example operation of the nuclear medicine diagnostic apparatus and a nuclear medicine image generating method according to the present embodiment is described.

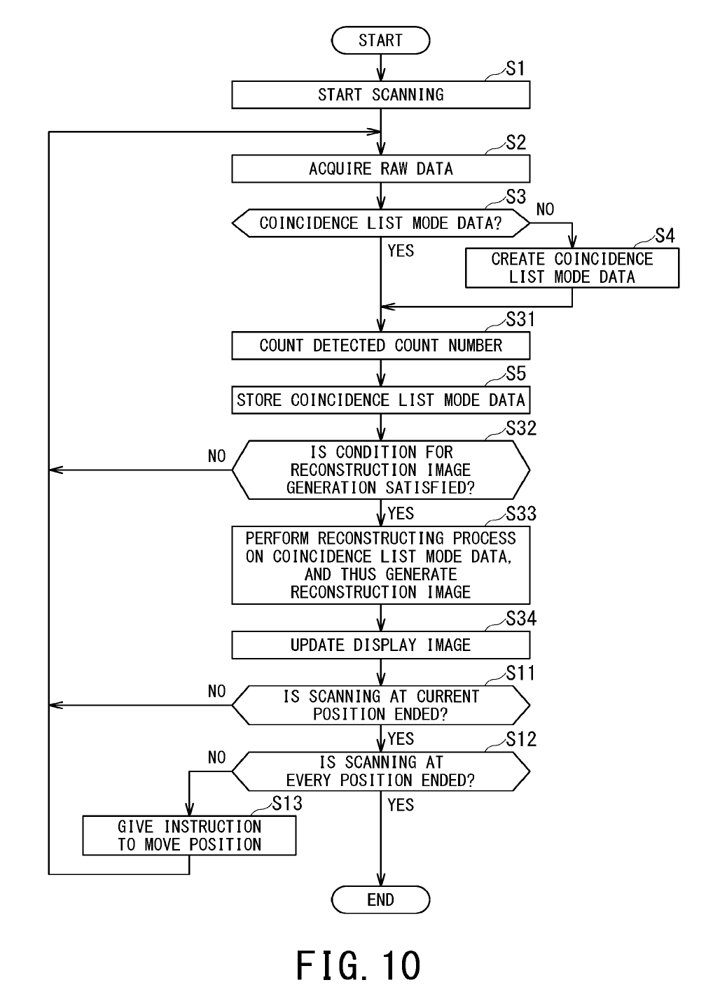

FIG. 10 is a flowchart illustrating a procedure when the processor of the processing circuitry 31B illustrated in FIG. 9 generates, in real time, a nuclear medicine image that enables the user to understand an occurrence position of an annihilation event through a high-speed image reconstructing process. In FIG. 10, reference signs of S with a number respectively denote steps in the flowchart.

This procedure is started at the time at which the patient O to whom a drug such as FDG has been administered is placed on the top plate 21. Steps equivalent to those in FIG. 6 are denoted by the same reference signs, and overlapping description is omitted.

In FIG. 10, description is given of an example case where scanning is performed according to the multi-head scan mode.

In Step S31, the counting function 61 counts a count number of coincidence count events received from the data collecting circuit 25. In a case where the count number is not used as the condition by the reconstruction controlling function 62, this step may not be executed.

In Step S32, the reconstruction controlling function 62 determines whether or not the condition necessary for the image reconstructing process is satisfied. If the condition necessary for the image reconstructing process is not satisfied, this procedure returns to Step S2. On the other hand, if the condition necessary for the image reconstructing process is satisfied, this procedure goes to Step S33.

In Step S33, the reconstruction image generating function 63 performs the image reconstructing process at high speed on the basis of coincidence count data (raw data stored in the raw data memory circuitry 51B), and thus generates a reconstruction image.

In Step S34, the display controlling function 46B expands the reconstruction image generated by the reconstruction image generating function 63, in the display image memory circuitry 54B, and displays the reconstruction image on the display 32.

Through the above-mentioned procedure, a nuclear medicine image that enables the user to understand an occurrence position of an annihilation event can be generated in real time through the high-speed image reconstructing process. Even if it is determined in Step S3 that the raw data is not coincidence list mode data, the raw data may be stored into the raw data memory circuitry 51B as it is. In this case, the coincidence list mode data may be created after raw data storage and before reconstruction image generation in Step S33.

The nuclear medicine diagnostic apparatus 10 according to the third embodiment can display a reconstruction image on the display 32 in a time shorter than the time required for scanning at one scan position in the multi-head scan mode and in a time shorter than the time required for a series of scanning operations in the continuous scan mode. Hence, the user can understand an occurrence position of a coincidence count event in real time on the basis of an image reconstructed at high speed, before an end of scanning at one scan position in the multi-head scan mode or before an end of scanning in the continuous scan mode.

Because the nuclear medicine diagnostic apparatus 10 according to the present embodiment can perform the image reconstructing process on raw data, the nuclear medicine diagnostic apparatus 10 according to the present embodiment can be applied to a PET apparatus that is not a TOF-PET apparatus.

With at least one of the above-described embodiments, at least one of a real-time image and a filter image can be displayed on the display 32 at least in the multi-head scan mode, and a nuclear medicine image that enables the user to understand an occurrence position of an annihilation event can be generated in real time in the multi-head scan mode.

The processing circuitry in the above-described embodiments 1-3 is an example of the processing circuitry described in the claims. In addition, the term “processor” used in the explanation in the above-described embodiments 1-3, for instance, a circuit such as a dedicated or general-purpose CPU (Central Processing Unit), a dedicated or general-purpose GPU (Graphics Processing Unit), an ASIC (Application Specific Integrated Circuit), a programmable logic device including an SPLD (Simple Programmable Logic Device) and a CPLD (Complex Programmable Logic Device) as examples, and an FPGA (Field Programmable Gate Array). A processor implements various types of functions by reading out programs stored in the memory circuit and executing the programs.

In addition, programs may be directly installed in the circuit of a processor instead of storing programs in the memory circuit. In this case, the processor implements various types of functions by reading out programs stored in its own circuit and executing the programs. Moreover, each function of the processing circuitry in the above-described embodiments 1-3 may be implemented by processing circuitry configured of a single processor. Further, the processing circuitry in the above-described embodiments 1-3 may be configured by combining plural processors independent of each other so that each function of the processing circuitry is implemented by causing each processor to execute the corresponding program. When plural processors are provided for the processing circuitry, a memory circuit for storing the programs may be provided for each processor or one memory circuit may collectively store all the programs corresponding to all the processors.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

Further, although an example of processing the steps of the flowchart is described in the embodiments in which each steps are time-sequentially performed in order along the flowchart, each step of the flowchart may not be necessarily processed in a time series, and may be executed in parallel or individually executed.

1. A nuclear medicine diagnostic apparatus comprising a processing circuitry configured to
acquire coincidence count data indicating an occurrence position of each of coincidentally counted pair-annihilation events, based on pieces of output data of a plurality of detectors that detect gamma rays emitted from radio isotopes administered to an object, and
each time a condition necessary for a filter process is satisfied, generate a filter image by performing the filter process on the coincidence count data.

2. The nuclear medicine diagnostic apparatus according to claim 1, wherein
each time a predetermined time necessary for the filter process elapses, the processing circuitry generates the filter image by performing the filter process on the coincidence count data.

3. The nuclear medicine diagnostic apparatus according to claim 1, wherein
each time a predetermined number of pieces of the coincidence count data are accumulated, the processing circuitry generates the filter image by performing the filter process on the coincidence count data.

4. The nuclear medicine diagnostic apparatus according to claim 1, wherein
each time the coincidence count data is acquired, the processing circuitry generates a real-time image by superimposing an image indicating the occurrence position of the annihilation event corresponding to acquired coincidence count data.

5. The nuclear medicine diagnostic apparatus according to claim 4, wherein
the processing circuitry displays the real-time image on a display each time the real-time image is generated.

6. The nuclear medicine diagnostic apparatus according to claim 1, wherein
the processing circuitry displays the filter image on a display each time the filter image is generated.

7. The nuclear medicine diagnostic apparatus according to claim 5, wherein
the processing circuitry displays an image obtained by superimposing the filter image and the real-time image on each other, on the display.

8. A nuclear medicine diagnostic apparatus comprising a processing circuitry configured to
acquire coincidence count data based on pieces of output data of a plurality of detectors that detect gamma rays emitted from radio isotopes administered to an object;
each time a condition necessary for an image reconstructing process is satisfied, generate a reconstruction image during scanning before an end of the scanning by performing the image reconstructing process based on the coincidence count data, and
display the reconstruction image each time the reconstruction image is generated.

9. The nuclear medicine diagnostic apparatus according to claim 8, wherein
each time a predetermined time elapses, the processing circuitry generates the reconstruction image by performing the image reconstructing process based on the coincidence count data collected in the predetermined time.

10. The nuclear medicine diagnostic apparatus according to claim 8, wherein
each time pair-annihilation events coincidentally counted by the plurality of detectors reach a predetermined number, the processing circuitry generates the reconstruction image by performing the image reconstructing process based on the predetermined number of pieces of the coincidence count data.

11. The nuclear medicine diagnostic apparatus according to claim 8, wherein
each time a time required for the image reconstructing process elapses, the processing circuitry generates the reconstruction image by performing the image reconstructing process based on the coincidence count data.

12. The nuclear medicine diagnostic apparatus according to claim 1, wherein
the processing circuitry associates bed position information with the coincidence count data.

13. A nuclear medicine image generating method, comprising:
acquiring coincidence count data indicating an occurrence position of each of coincidentally counted pair-annihilation events, based on pieces of output data of a plurality of detectors that detect gamma rays emitted from radio isotopes administered to an object; and
each time a condition necessary for a filter process is satisfied, generating a filter image by performing the filter process on the coincidence count data.
